(19) United States
(12) Patent Application Publication
(10) Pub. No.: US 2010/0004629 A1
(43) Pub. Date: Jan. 7, 2010

(54) APPARATUS AND METHODS TO IMPLEMENT A VERSATILE LIQUID STORAGE AND DELIVERY MECHANISM

(75) Inventor: Eduardo P. Dolhun, San Francisco, CA (US)

Correspondence Address: PERKINS COIE LLP, P.O. BOX 1208, SEATTLE, WA, 98111-1208, US

(73) Assignee: Drip Drop Solutions, Inc.

(21) Appl. No.: 12/495,475

(22) Filed: Jun. 30, 2009

Related U.S. Application Data
(60) Provisional application No. 61/078,280, filed on Jul. 3, 2008.

(51) Int. Cl. A61J 1/05; B65D 8/04; A61M 39/00; A61J 1/20

(52) U.S. Cl. 604/516; 220/669; 604/403; 604/257; 604/246; 604/414

(57) ABSTRACT

A container shaped in the form of a teardrop is used interchangeably in upright and inverted orientations. The container is used in an upright orientation to allow a person to directly drink liquids. In the inverted orientation, the container is mounted at a height over the user and allows versatile and controlled administration of liquids even to bedridden patients. The controlled administration provides the advantages of precise and measured dispensation of liquids, on-site or remote monitoring of dispensation, and control of dispensation by a patient or a care-giver. This type of liquid dispensation is not as invasive as other medical treatments (e.g., intravenous treatment), and therefore can be employed both at home and at hospitals with comparable effectiveness.

### CROSS-REFERENCED TO RELATED APPLICATION

This application claims priority to U.S. Provisional Application No. 61/078,280, filed Jul. 3, 2008, which is hereby incorporated by reference in its entirety.

### FIELD OF INVENTION

The invention relates in general to apparatus and methods for implementing a versatile liquid storage and delivery mechanism. Specifically, the invention relates to apparatus and methods for implementing a liquid container that can be interchangeably used in upright or suspended manners, thus allowing it to be used for example as regular water-bottles in domestic settings or for controlled oral administration of liquids in hospital-type settings.

### BACKGROUND OF THE INVENTION

Dehydration is a condition that occurs when a person loses excessive amounts of fluids. Mild to moderate levels of dehydration may occur due to strenuous physical activities, gastrointestinal disorders, inadequate consumption of liquids, etc. Moderate to severe levels of dehydration may occur after a person undergoes surgeries or after a person undergoes radiation therapy or chemotherapy.

While mild levels of dehydration may not require medical attention, moderate to severe levels of dehydration are cured by providing medical treatment. This medical treatment involves intravenous injection (IV treatment) of fluids. This invasive IV treatment is offered as the solution for almost all forms of moderate to severe levels of dehydration. Because IV treatment requires administration by qualified medical personnel, treatment of moderate and severe levels of dehydration takes up valuable medical resources (e.g., emergency room bed spaces, availability of medical staff, etc.). This leads to increase in medical costs for both the patient and the hospital.

While IV treatment may be the only option for treatment in critically severe cases of dehydration, most moderate levels and non-critical severe levels of dehydration may successfully be treated by oral administration of fluids. In addition to treatment for dehydration, oral treatment solutions are considered ideal in other medical ailments, such as in the treatment of electrolyte imbalance or administration of antibiotics to a patient. However, such oral treatment is not completely effective because oral treatment does not offer some of the unique advantages offered by an IV treatment system.

Despite its excessive cost and resulting inefficiencies, IV treatment is preferred over oral treatment because it allows a caregiver to control the rate at which liquids are injected. In addition, IV treatment allows a person to monitor the amount of liquids dispensed, allows for remote monitoring of the injection of the fluids, and also allows precise metering of the liquids. Prior art solutions to oral treatment invariably require a person to carry a container containing the required liquids and consume the liquids directly from such containers. Such prior art solutions do not allow for precise metering of the liquids, do not provide means to control delivery of the liquids, and do not provide easy means for a third person to monitor the dispensation of the liquids. Prior art approaches also do not effective provide means by which oral treatment can be administered to people with limited arm movement (e.g., bedridden patients, post-surgery patients, etc.). Additionally, prior art approaches are limited in scope, meaning that they can either be used for oral delivery of liquids or for intravenous delivery of liquids, offering limited or no versatility in the usage of the liquid delivery mechanism.

### SUMMARY OF THE INVENTION

Apparatus and methods to implement a versatile liquid storage and delivery mechanism are presented. In one embodiment, the present invention provides an effective alternative to IV treatment in some moderate to severe cases of dehydration. In another embodiment, the container can be used for enteral delivery of liquids to hospitalized patients, including gastric and nasogastric delivery of liquids. In one embodiment, the present invention includes using a versatile liquid container that is shaped in the form of a teardrop. The teardrop shape has a rounded lower portion and a tapered upper portion. This container is used to store and deliver liquids essential to the treatment of dehydration.

In one example, the container can be carried and used like a regular water-bottle in its upright orientation, where the rounded lower portion of the container points downward. However, when there is a need for oral administration of liquids through means similar to an IV treatment (e.g., oral treatment for severely dehydrated patients in a hospital, enteral delivery of liquids, etc.), the same container can be used in an inverted orientation. In its inverted position, the lower rounded portion of the container points upward, and the structure of the container allows it to be suspended at a height above ground level. In this example, a feeding tube is used to deliver the liquids from the container to the patient in need of the oral treatment. In one embodiment, the feeding tube is provided with control mechanisms to allow the patient or a care-giver to meter and control the rate at which the liquids are delivered to the patient. Examples of the control mechanism include hand-operated pumps, mechanical controls, or electronic control systems. The container can be mounted from provisions on hospital beds, wheelchairs, gurneys or IV stands, etc., and can therefore be employed in a variety of settings.

In another example, the container can be used as an alternative to IV treatment to provide medication where oral or enteral administration of medicines would be more effective (e.g., administration of antibiotics, treatment of electrolyte imbalance, etc.). Consequently, the present invention can be employed not only for the purposes of dehydration treatment, but also for any treatment or application that requires administration of liquids.

Thus, the present invention offers several advantages, including freeing up hospital care givers (e.g., nurses) from having to hand-deliver liquids to the patients, allowing the patient or the care-giver to control the quantity and rate at which liquids are delivered, preventing accidental choking or excessive dispensation of liquids, etc. Additionally, since this procedure does not require invasive injections (of the type required in IV treatments), the oral administration can be done at home, thus saving costs and freeing up hospital resources. Because the container can be used interchangeably in inverted and upright orientations, the same container can be used in both hospitals and at home, thus further improving the effectiveness of the oral treatment.

In another embodiment, this invention provides methods of efficient packing and storing of the containers. In this embodiment, a plurality of the containers is packed in a saw-tooth pattern. Here, a first row of containers is placed in the upright position over which a second row of containers is placed in the inverted position. In this embodiment, the saw-tooth arrangement ensures minimal void space between the rows of containers. This leads to increased shipping efficiency by minimizing the size and volume of the overall shipments, and also ensures reduced chances of damage to the containers during shipment.

### BRIEF DESCRIPTION OF DRAWINGS

These and other objects, features and characteristics of the present invention will become more apparent to those skilled in the art from a study of the following detailed description in conjunction with the appended claims and drawings, all of which form a part of this specification. In the drawings:

FIG. 1 illustrates one embodiment of a tear-drop shaped container;

FIG. 2 provides an embodiment of the container showing different examples of openings to allow bidirectional flow of liquids;

FIG. 3 depicts various embodiments of the container with provisions that allow it to be seated firmly on a ground surface;

FIG. 4 shows an embodiment of the container used in an inverted orientation;

FIG. 5 provides an example of how the inverted container can be suspended at a height above ground level;

FIG. 6 illustrates one embodiment of how the container can be used for oral or enteral administration of liquids;

FIG. 7A depicts various embodiments showing means by which the oral administration of liquids can be controlled;

FIG. 7B depicts various additional embodiments showing means by which the oral administration of liquids can be achieved;

FIG. 7C illustrates the use of additive inlets to allow materials to be added into the container;

FIG. 7D provides examples of pellets that are be used for standardized addition of materials through the additive inlet;

FIG. 8 provides several examples of different design versions of the container;

FIG. 9 provides an embodiment of an overall method for interchangeable use of the versatile liquid storage and delivery mechanism;

FIG. 10 is a flow chart depicting one embodiment by which the oral administration of liquids can be controlled, monitored, or metered;

FIG. 11 is a flow chart illustrating one embodiment of a method for efficient packing of a plurality of the containers; and

FIG. 12 is a picture providing an example of how the plurality of containers can be efficiently packed together.

### DETAILED DESCRIPTION OF THE INVENTION

The present invention may be embodied in several forms and manners. The description provided below and the drawings show exemplary embodiments of the invention. Those of skill in the art will appreciate that the invention may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

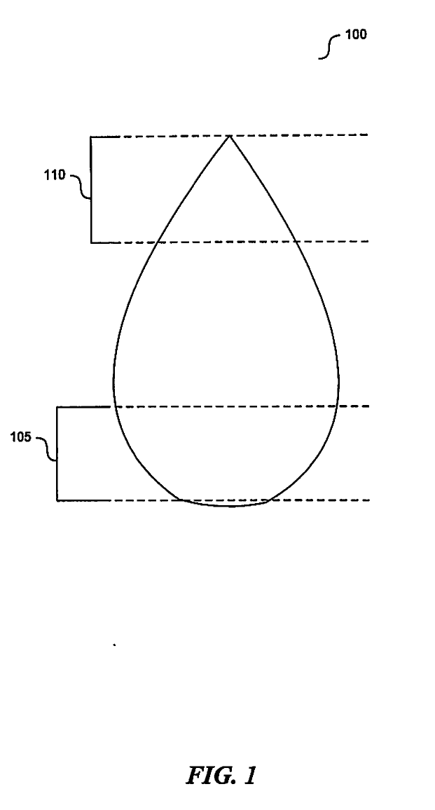

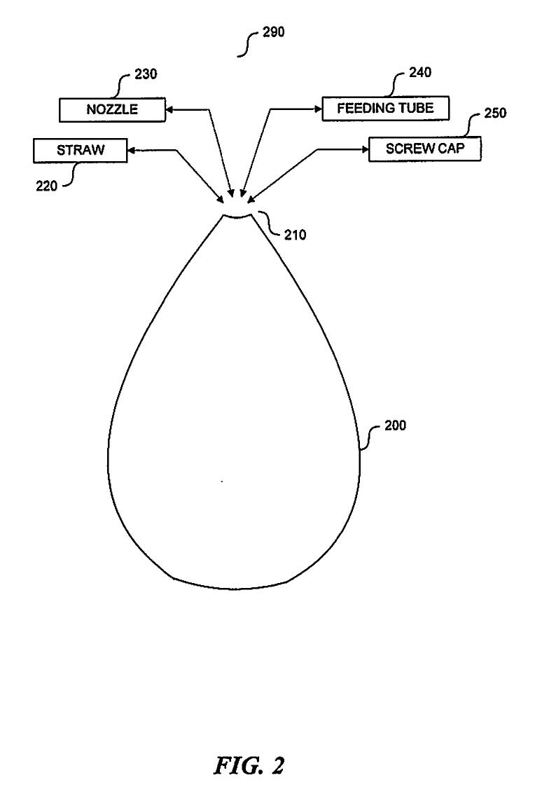

FIG. 1 shows one embodiment of a versatile liquid storage and delivery apparatus. In this embodiment, the apparatus is a container 100 in the shape of a teardrop. The tear drop is used for illustration of one embodiment of the invention, and it is understood that the container 100 can be realized in other shapes or forms to enable versatile storage and delivery of liquids. Additionally, the term teardrop is not used in reference to one particular form or shape, and is not restricted to the shape, geometry, or dimensions of the embodiment provided in FIG. 1. The shape of the container 100, as signified by the teardrop shape, can include any shape that has a broader and rounded or curve-shaped lower portion 105 (e.g., the base of a rounded triangle, a semi-circular or hemispherical shape, etc.) that is larger in dimension than the upper portion 110 of the container 100. The term teardrop further signifies that the container 100 has a narrower and tapered upper portion 110 as exemplified in FIG. 1. This container 100 can be used to store or deliver fluids by means of an opening as described in FIG. 2 (described in detail further below). In one embodiment, the container 100 is made from one or more hard materials. Examples of hard materials include wood, metal, plastic, etc. In another embodiment, the container 100 is made from one or more soft materials. Examples of soft materials include synthetic fabrics, soft plastic, rubber, etc.

FIG. 2 shows several examples 290 of how an opening is used for bidirectional flow of liquids to and from the container 200. FIG. 2 shows one embodiment of the container 200, indicating an opening 210 located at or near the apex of the upper portion of the container. The opening 210 can be realized in any number of embodiments, depending on the manner in which the container is intended to be used by a user. Some examples of the opening include a drinking straw 220, a nozzle 230, a feeding tube 240 to allow flow of liquids in or out of the container. The opening may also be realized in the form of a selectively sealable cap (e.g., a screw cap 250) to escape of liquids from container 200.

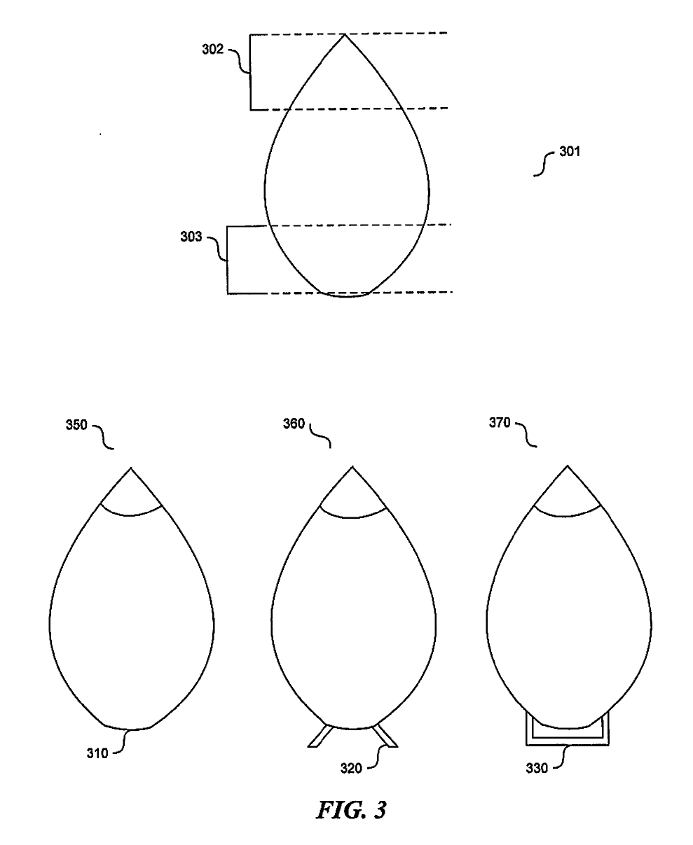

FIG. 3 shows several examples of how the container 301 can be allowed to be seated in an upright orientation. The original shape of the container as shown in 301 has an upper portion 302 and a lower portion 303. An upright orientation for the container indicates that the lower portion 303 of the container 301 points downward and the upper portion 302 of the container 301 points upward. When used in such an upright orientation, the container 301 can be allowed to rest or be seated on a solid surface. When the lower portion 303 of the container 301 is rounded, some alterations or additional provisions can be provided to the container 301 to allow it to be seated on a solid surface. In one example 350, the base 310 of the container can be a flattened surface. In another example 360, retractable stands 320 are provided to the container. In yet another example 370, a handle-like provision 330 is provided such that the container can be placed on a solid surface. Such additional provisions can be made of the same material as the container, or can be made using other materials such as metal, plastic, wood, etc.

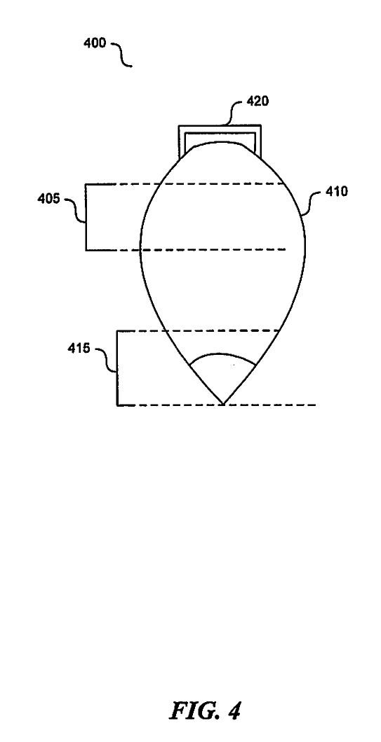

FIG. 4 illustrates an embodiment where the container is in an inverted orientation 400. In this inverted orientation, the lower portion 405 of the container 410 points upward and the upper portion 415 of the container 410 points downward. The container 410 has a placement provision 420, similar to the example 370 shown in FIG. 3. In this embodiment, this placement provision 420 can be a handle-like attachment that enables the container 410 to be suspended at a height above the ground level.

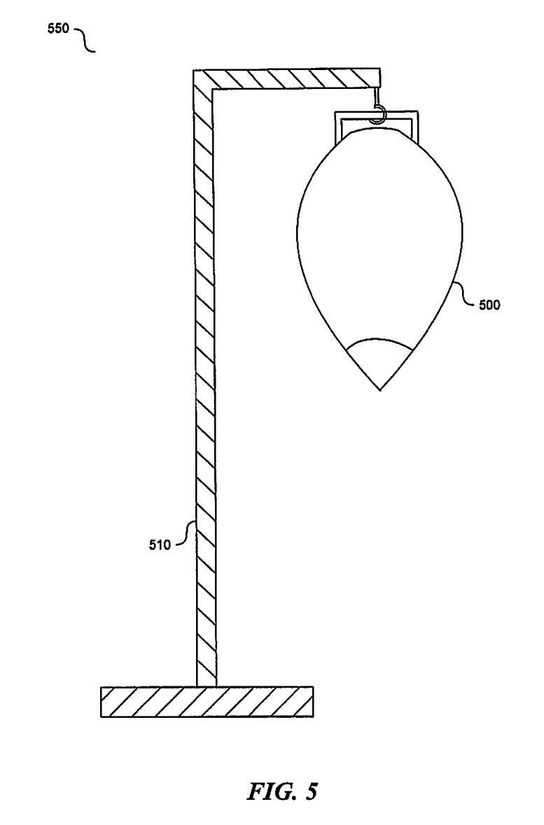

FIG. 5 illustrates an embodiment showing how the container 500 can be suspended from a height above the ground level. In this embodiment 550, the container 500 is mounted from a support source such that the container can be suspended at a height above ground level. Examples of such support sources include IV stands 510 that are normally available in hospitals that support suspension of bags dispensing intravenous fluids, a mounting provision or stand 520 attached to a bed, a mounting provision or stand attached to a gurney 530, a mounting provision or attached to a wheel chair 540, etc. The height of these stands may be altered to adjust to a particular requirement or the comfort level of the user.

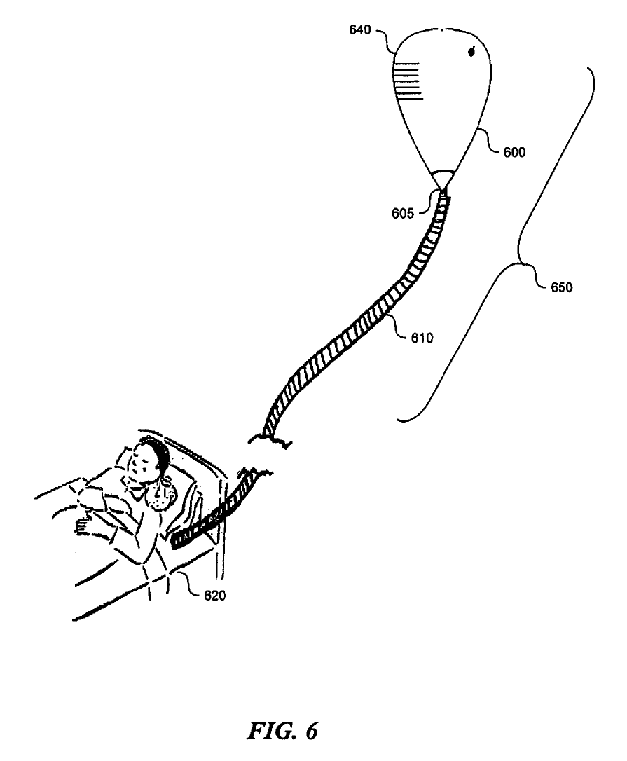

FIG. 6 shows an embodiment of a liquid dispenser for the container 600 in inverted orientation, where a feeding tube 610 is attached to the container 600 to allow liquids to flow out of the container 600. One end of the feeding tube 610 is located at or near the opening 605 of the inverted container 600. The other end of the attachable tube 610 can be made available to the receiver 620 of the liquids (e.g., a hospital patient). In one example, this other end of the feeding tube 610 is fitted with a mouthpiece to allow the fluids to be easily dispensed to the receiver 620 of the liquids. The mouthpiece is also provided with suck or no-suck options, thus making it easy for the receiver 620 to receive the liquids only when desired. In other embodiments, the feeding tube can be used for enteral delivery of liquids, including gastric delivery 660 and nasogastric delivery 670 of liquids. The feeding tubes 610 could be or could resemble the intravenous drip tubes used in hospitals. The feeding tube 610 can be obtained in several standard length options or can also be obtained in customized lengths. The combination of the attachable tube 610 and the inverted container 600 provides a liquid dispensing system 650 that is used for a versatile suite of applications, including oral re-hydration therapy, oral dispensation of liquids in hospitals, gastric enteral dispensation or delivery of liquids, nasogastric enteral dispensation or delivery of liquids, etc.

In one embodiment of the container is provided with measurement hashes 640 such that a user or a caregiver can precisely observe, monitor, and meter the quantity of liquids received by the user 620.

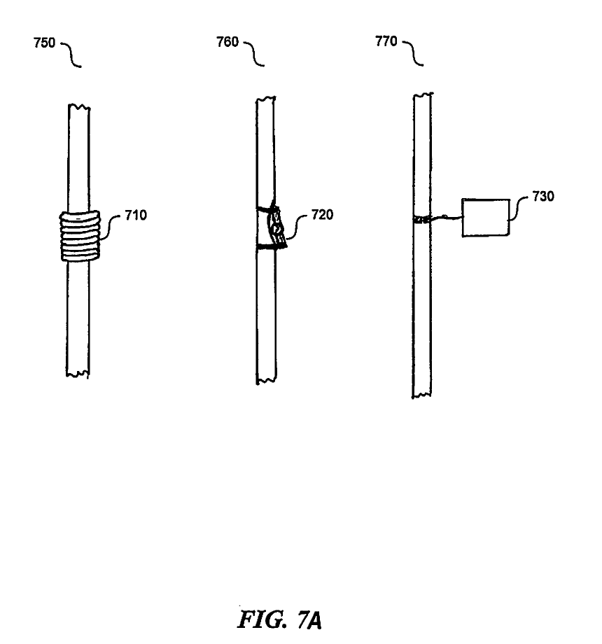

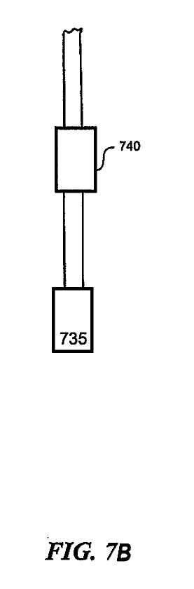

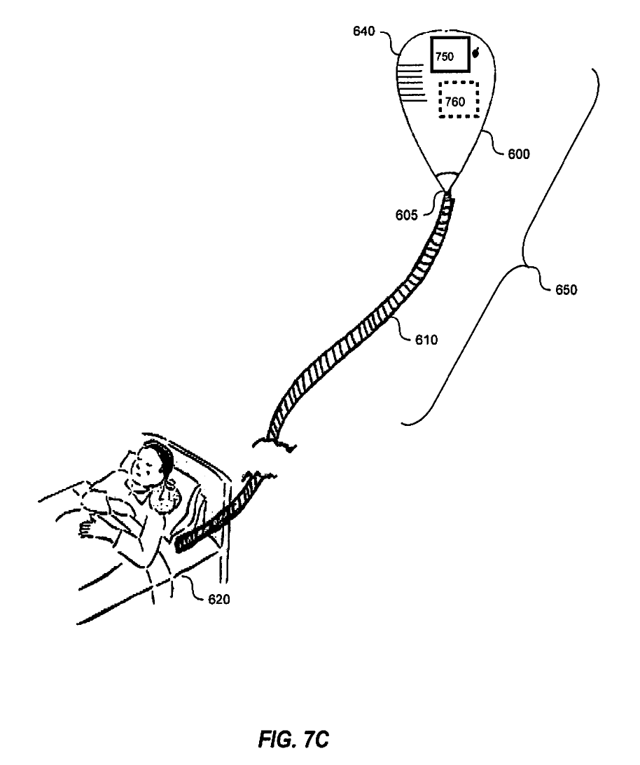

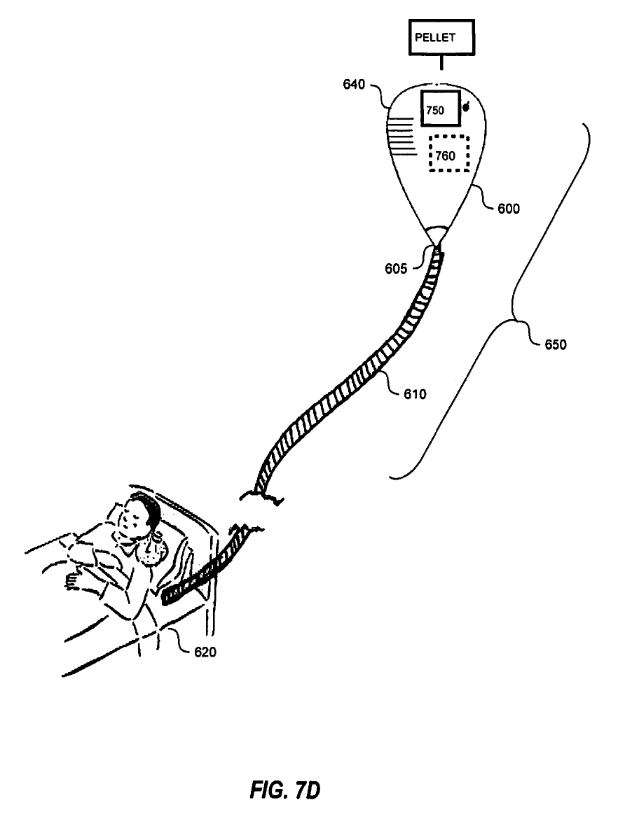

FIG. 7A shows several embodiments of means to control and monitor delivery of liquids using the liquid dispensing system 650 explained in FIG. 6. In one embodiment 750, the feeding tube uses a squeezer attachment 710, which can operate as a hand-pump. By providing this, a user (e.g., a hospital patient being treated for dehydration) of the liquids dispensing system (650 from FIG. 6) can control the rate at which the liquids are dispensed or even the quantity of liquids dispensed. This provides the advantages of freeing others (e.g., nurses) from personally having to deliver the liquids, preventing spillage of liquids, ensuring that the user stops receiving liquids when the user falls unconscious, preventing the user from choking or imbibing excessive amount of liquids, etc.

FIG. 7A also provides additional examples of how the liquid dispensing system can be controlled and monitored. In one embodiment 760, the feeding tube is fitted with a mechanical control 720 that can be used to adjust the rate and quantity of liquids dispensed. This mechanical control 720 is similar to mechanical controls available on the intravenous drip delivery systems, and such a control will be readily apparent to a person skilled in the art. FIG. 7 also provides another embodiment 770, where the liquid dispensation can be controlled or monitored using an electronic control system 730. Such an electronic control system 730 includes systems widely used in hospitals to control the delivery of liquids (e.g., TBD).

As shown in FIG. 7B, in one embodiment, the receiving end of feeding tube is fitted with a mouthpiece 735 to allow a user to drink liquids from the feeding tube. Additionally, the feeding tube is also optionally fitted with a flow control provision to prevent the escape of fluids through the feeding tube when the feeding tube is not in use. Examples of the flow control provision include valves 740 with open and shut mechanisms, and other such provisions widely available in the art.

FIG. 7C also provides an example of an embodiment where the liquid dispensing system (650 from FIG. 6) is provided with an additive inlet. In one embodiment, the additive inlet is an opening 750 on the exterior of the container, allowing additive materials (e.g., soluble medication, minerals, soluble food supplements, etc.) to be added to the contents of the container without disturbing the feeding tube. This opening optionally includes a seal that can be unsealed when additive materials are added in to the container and sealed after the additive materials are added in. In another embodiment, the additive inlet is a injectable surface 760 that allows additive materials to be injected directly into the container using a needle and syringe type of setup.

FIG. 7D illustrates the use of standard sized pellets to add the additive materials into the container. The pellets are available in a variety of standard sizes (e.g., 100 cc, 1000 cc, etc.) and are adapted to the additive inlet (750 or 760 from FIG. 7C) of the container. In one embodiment, the pellet comprises an unsealable opening such that the contents of the pellets can be squeezed into the container through the opening 750 on the container. In another embodiment, the pellet comprises a needle attachment such that the needle can be pierced through the injectable surface 760 to allow the contents of the pellets to be added in to the container.

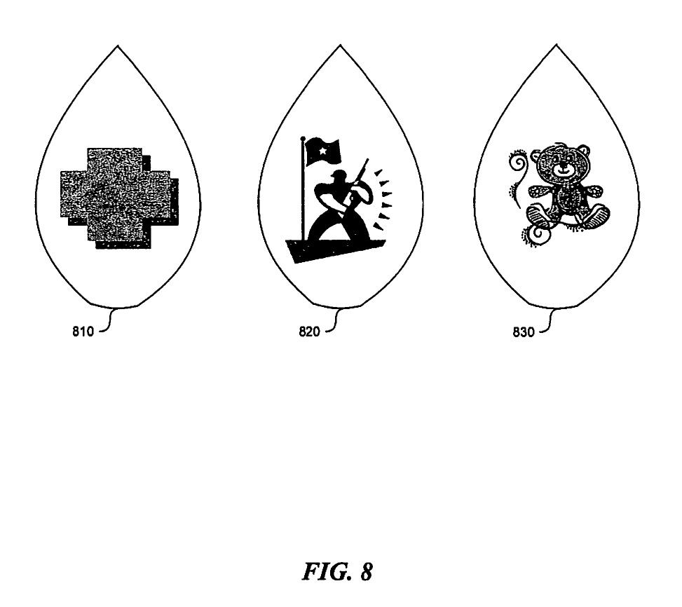

The container 100 shown in FIG. 1 can be made available in a variety of colors and sizes. Different versions of the container can also be made for use in different settings. For example, a hospital version of the container 100 can be made, where the container 100 allows for sterilization and reuse procedures. Additional embodiments may be made as shown in FIG. 8, where the container 100 can have different designs on its exterior (e.g., containers as shown in 810, 820, and 830), thus ensuring that they appeal to different demographics.

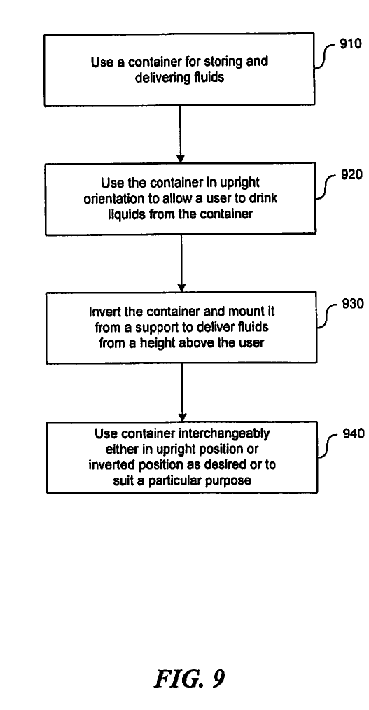

FIG. 9 is a flowchart depicting an embodiment of an overall method for implementing a versatile liquid storage and delivery mechanism. In this embodiment, a container shaped in the form of a teardrop is used for the purpose of storing and delivering fluids, as indicated by the flow chart element 910. The term teardrop is used merely in a descriptive manner, and not in any restricting manner to confine the structure of the container to one particular shape or form. Furthermore, the term teardrop generally indicates that the container has a broader and rounded lower portion, and a narrower and tapered upper portion. In one embodiment, the container is first used in an upright orientation as provided in flow chart element 920, where the upper portion of the container points upward and the lower portion of the container points downward. This upright orientation allows a person to drink liquids directly from the container.

As further shown in FIG. 9, the container can then be inverted to be used in an inverted orientation, where the lower portion of the container points upward, and the upper portion of the container points downward as shown in element 930 of the flowchart. In one embodiment, the container is used by suspending it from a height above the ground level, such that liquids can be delivered to a person by means of gravity. The container can then be brought back to its upright position at any time after the need for dispensing fluids at a height over ground level has dissipated. Thus, as indicated in element 940 of the flowchart, the container can be used interchangeably in at least the upright and inverted positions as desired by the person receiving the liquids.

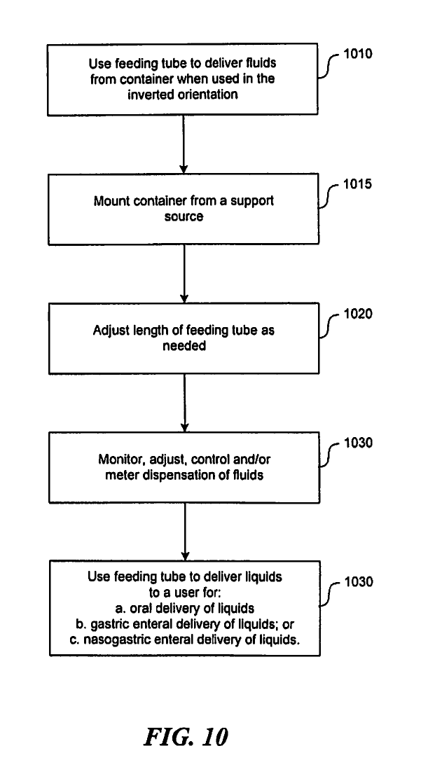

FIG. 10 is a flowchart that describes the method by which the container can be used in an inverted orientation. In one embodiment, a feeding tube is first used to deliver liquids from the container, as depicted in step 1010 of the flowchart. The length of this attachable tube can be adjusted 1020. In the next step 1030, the dispensation of the liquids can be controlled, monitored, or metered by the patient or a third person (e.g., a caregiver) by using a controller mechanism. Examples of controller mechanisms include hand operated pumps, mechanical controller devices, and electronic controller devices. Examples of mechanical controller devices include knob-like structures that are commonly found in IV tubes (used to control the rate of flow of intravenous liquids). Similarly, there are several electronic controller devices available commercially that a person skilled in the art would be easily able to obtain. These embodiments can allow the person (e.g., a patient who is the receiver of the fluids) to control or monitor the delivery of liquids. Additionally, a third-party person (e.g., a nurse taking care of the patient) can also control, meter, and monitor the delivery of the liquids.

In one illustrative application of this embodiment, the container can be used in the inverted position in a hospital setting to provide fluids to a person (e.g., a patient) suffering from moderate to severe dehydration. In such an application, a care-giver (e.g., a nurse or a doctor) can monitor and control the amount of fluids the patient should imbibe by using the method illustrated in this embodiment. When the patient has sufficiently recovered and is ready to be discharged from the hospital setting, the container can be brought back to its upright position. The container can be given to the patient to take home such that the patient can continue receiving the required liquids from the same container even outside of the hospital setting. Other examples of applications where the container is used in inverted orientation include nasogastric enteral delivery of liquids and gastric enteral delivery of liquids to a patient in a hospital setup.

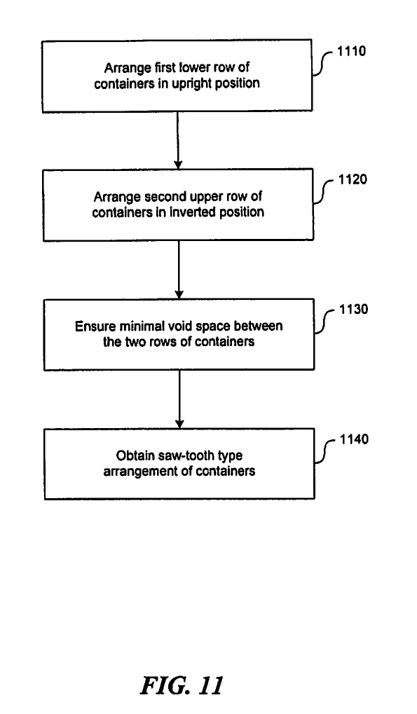

FIG. 11 is a flow chart depicting a method for arranging several containers in a shipping or storage box. In one embodiment, a row of containers is first placed such that all the containers in that row are in the upright orientation, as indicated in step 1110 of the flowchart. A second row of containers is then placed over the first row, where all the containers in this second row are in the inverted orientation, as indicated in step 1120 of the flowchart. The second row is placed over the first row in a manner that ensures minimal void space between the two rows of containers, as indicated in step 1130. This method of arrangement results in the final step 1140, providing a saw-tooth type of arrangement.

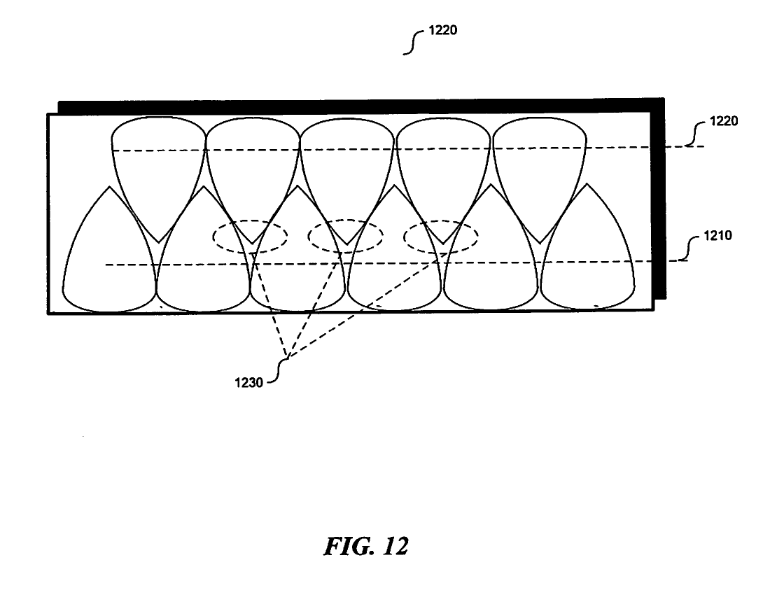

An example 1200 of this saw-tooth type of arrangement is illustrated in FIG. 12. Here, 1210 is the first row of containers in upright orientation, and 1220 is the second row of containers in inverted position. This type of placement is done while ensuring that there is minimal void space 1230 between the two rows of containers. This method of packaging improves the efficiency of shipping the containers by reducing the overall shipping volume. This method also ensures that there is minimal void space between the rows of containers, thereby reducing chances of damage during shipping. Additionally, this method of packing ensures that the containers preserve their shape when the containers are shipped, further improving the shipping efficiency.

A first embodiment of the techniques described herein includes an apparatus for implementing a versatile liquid storage and delivery mechanism, the apparatus comprising a container; said container having an upper portion and a lower portion; said container having an opening for bidirectional flow of liquids; said container used interchangeably in an upright orientation or in an inverted orientation, wherein, said container when in said upright orientation is arranged for a user to carry said container and is convenient for said user to drink liquids from said container; and said container when in said inverted orientation is arranged for delivering liquids to said user when mounted on a support source and is suitable for at least one of oral delivery of liquids; or enteral delivery of liquids; and said container having a placement provision, wherein said placement provision is suitable for placing said container on a solid surface in said upright orientation, and wherein said placement provision is suitable for mounting said container on said support source in said inverted orientation.

Wherein, in the first embodiment said container is in the shape of a tear drop; said upper portion is tapered and narrow, said lower portion is rounded and broader than said upper portion; said upper portion points upward and said lower portion points downward when said container is in said upright orientation; said upper portion points downward and said lower portion faces upward when said container is in said inverted orientation; said enteral delivery of liquids includes at least one of: gastric delivery of liquids, or nasogastric delivery of liquids; said opening includes at least one of a drinking straw, a nozzle, or a feeding tube; said opening includes a sealing provision to prevent liquids from escaping said container; said placement provision includes at least one of flattening of said lower portion enabling said container to be placed on said solid surface when said container is in said upright orientation, an external attachment enabling said container to be placed on a solid surface when said container is in said upright orientation, or mounted on said support source when said container is in said inverted orientation; said container in said upright orientation is suitable for carrying by hand by said user; said support source enables said container to be mounted at a height above said user, said support source including at least one of an IV stand, a mounting provision on a bed, a mounting provision on a gurney, or a mounting provision on a wheel chair; said feeding tube is used when said container is in said inverted orientation, and includes any mechanism that enables liquids to be delivered from said container to a location at which said user can receive liquids; said feeding tube is available at a variety of standardized or customized lengths; said feeding tube includes one or more controlled-delivery devices; said controlled-delivery device includes at least one of a hand-controlled squeezer, a hand-controlled pump, a mechanical controller, or an electronic controller; said controlled-delivery device enables said user to do at least one of control rate of delivery of liquids, monitor delivery of liquids, or meter delivery of liquids; said controlled-delivery device enables a caregiver to do at least one of control rate of delivery of liquids to said person, monitor delivery of liquids to said person, or meter delivery of liquids to said person; a first end of said feeding tube is at or near said opening of said container; a second end of said feeding tube has a mouthpiece; said mouthpiece has suck and no-suck options; said mouthpiece further includes a flow control provision to prevent escape of liquids when said feeding tube is not in use; said container has an additive inlet that enables adding one or more additives to contents of said container; said additive inlet includes at least one of a sealable additive opening on the exterior of said container, said sealable additive opening is unsealed to add said additives and said sealable additive opening is sealed after said additives are added to contents of said container, or an injectable area on the exterior of said container, wherein a needle is pierced through said injectable area to enable said additives to be injected into said container; standardized pellets are used for adding said additives to contents of said container; each of said standardized pellets contains a specific quantity of said additives; said standardized pellet includes a pellet opening to allow flow of liquids out of said standardized pellet, said pellet opening including one of a nozzle, wherein said nozzle is adapted to fit within said sealable additive opening of said container, or a needle arrangement that can be used to pierce through said injectable area of said container; said container has a measurement provision to indicate quantity of liquids within said container; said measurement provision includes providing measurement markings on or within said container; said container is available in several color option; said container is available in several size options; said container is available in several design options; said container is made using a hard material; said container is made using a soft material.

A second embodiment of the techniques described herein includes a method for implementing a versatile liquid storage and delivery mechanism, the method comprising using a container to store or deliver liquids, wherein said container has an upper portion and a lower portion, using said container interchangeably in an upright orientation or in an inverted orientation, wherein using said container in said upright orientation enables a person to directly drink liquids from said container, using said container in said inverted orientation includes mounting said container from a support source, enabling said container to be used for at least one of oral delivery of liquids, or enteral delivery of liquids, and using a placement provision, wherein said placement provision enables said container to be placed on a solid surface in said upright orientation, and wherein said placement provision enables said container to be mounted from said support source in said inverted orientation.

Wherein, in the method of the second embodiment, said container is in the shape of a tear drop; said upper portion is tapered and narrow, and said lower portion is rounded and broader than said upper portion; said upper portion points upward and said lower portion points downward when said container is used in said upright orientation, and wherein said upper portion points downward and said lower portion points upward when said container is used in said inverted orientation; said container has an opening allowing bidirectional flow of liquids, said opening including at least one of, a drinking straw, a nozzle, or a feeding tube; said controlling device enables at least one of, metering dispensation of liquids, controlling rate of dispensation of liquids, or monitoring dispensation of liquids.

Wherein, the method of the second embodiment further comprises arranging said containers in a saw-tooth arrangement; wherein in said saw-tooth arrangement, a first row of said containers is placed in said upright orientation, and a second row of said containers is placed over said first row of said containers in said inverted orientation such that there is minimal void space between said first row and said second row of said containers; said arranging in said saw-tooth arrangement is done when a plurality of said containers are packed in a container; said arranging in said saw-tooth arrangement minimizes damage to said containers; said arranging in said saw-tooth arrangement improves efficiency of shipping said plurality of containers.

A method of a third embodiment of the techniques disclosed herein includes a method for implementing a versatile liquid delivery mechanism, the method comprising using a container interchangeably in an upright orientation or an inverted orientation, using said container in said upright orientation for delivering liquids to a person, wherein said using said container in said upright orientation enables a person to directly drink liquids from said container, using said container in said inverted orientation for delivering liquids to a person, wherein using said container in said inverted orientation includes mounting said container on a support source such that said container is suitable for at least one of oral delivery of liquids, or enteral delivery of fluids, using a feeding provision to enable liquids to be delivered to said person, using a flow measuring method to measure and quantify delivering of liquids, using a flow controlling method to allow said person or a third person to control delivering of liquids.

Wherein, in the method of the third embodiment, said flow measuring method includes using said containers of a specific capacity; said flow measuring method includes using measurement indicators on or within said container; said enteral delivery includes gastric delivery of liquids; said enteral delivery includes nasogastric delivery of liquids; using said feeding provision includes using at least one of a nozzle, a feeding tube, or a drinking straw; using said container in said inverted position comprises mounting said container on said support source at a height above said person; using said container in said inverted position further comprises delivering liquids by means of gravity through said feeding provision; said mounting said container at a height includes mounting said container from an IV stand, mounting said container from a stand attached to a bed, mounting said container from a stand attached to a gurney, or mounting said container from a stand attached to a wheel chair; using said container in said upright position includes hand-carrying said container; said feeding provision has a flow control provision to prevent escape of liquids when said feeding provision is not used by said person; the method further comprising using an additive inlet to add one or more additives to contents of said container; said adding one or more additives includes at least one of adding a base, adding an acid, adding a prescriptive or over-the-counter drug, adding electrolytes, adding vitamins, or adding sweeteners.

A fourth embodiment of the techniques introduced here includes a method for implementing a versatile liquid delivery mechanism, the method comprising using a container interchangeably in an upright orientation or an inverted orientation, using said container in said upright orientation for delivering liquids to a person, wherein said using said container in said upright orientation enables a person to directly drink liquids from said container, using said container in said inverted orientation for delivering liquids to said person, wherein using said container in said inverted orientation includes mounting said container from a support source, and using a placement provision, wherein said placement provision enables said container to be placed on a solid surface in said upright orientation, and wherein said placement provision enables said container to be mounted from said support source in said inverted orientation.

A fifth embodiment of the techniques introduced herein includes an apparatus for implementing a versatile liquid storage and delivery mechanism, the apparatus comprising a container used interchangeably in an upright orientation or in an inverted orientation, wherein said container when in said upright orientation is arranged for a user to carry said container and is convenient for said user to drink liquids from said container, and said container when in said inverted orientation is arranged for delivering liquids to said user when mounted on a support source; and said container having a placement provision, wherein said placement provision is suitable for placing said container on a solid surface in said upright orientation, and wherein said placement provision is suitable for mounting said container on said support source in said inverted orientation.

In addition to the above mentioned examples, various other modifications and alterations of the invention may be made without departing from the invention. Accordingly, the above disclosure is not to be considered as limiting and the appended claims are to be interpreted as encompassing the true spirit and the entire scope of the invention.

I claim:

1. An apparatus for implementing a versatile liquid storage and delivery mechanism, the apparatus comprising:
a container;
said container having an upper portion and a lower portion;
said container having an opening for bidirectional flow of liquids;
said container used interchangeably in an upright orientation or in an inverted orientation, wherein:
said container when in said upright orientation is arranged for a user to carry said container and is convenient for said user to drink liquids from said container; and
said container when in said inverted orientation is arranged for delivering liquids to said user when mounted on a support source and is suitable for at least one of:
oral delivery of liquids; or
enteral delivery of liquids; and
said container having a placement provision, wherein said placement provision is suitable for placing said container on a solid surface in said upright orientation, and wherein said placement provision is suitable for mounting said container on said support source in said inverted orientation.

2. An apparatus for implementing a versatile liquid storage and delivery mechanism, as recited in claim 1, wherein:
said container is in the shape of a tear drop;
said upper portion is tapered and narrow;
said lower portion is rounded and broader than said upper portion;
said upper portion points upward and said lower portion points downward when said container is in said upright orientation;
said upper portion points downward and said lower portion faces upward when said container is in said inverted orientation.

3. An apparatus for implementing a versatile liquid storage and delivery mechanism as recited in claim 1, wherein said enteral delivery of liquids includes at least one of:
gastric delivery of liquids; or
nasogastric delivery of liquids.

4. An apparatus for implementing a versatile liquid storage and delivery mechanism as recited in claim 1, wherein said placement provision includes at least one of:
flattening of said lower portion enabling said container to be placed on said solid surface when said container is in said upright orientation; or
an external attachment enabling said container to be:
placed on a solid surface when said container is in said upright orientation; or
mounted on said support source when said container is in said inverted orientation.

5. An apparatus for implementing a versatile liquid storage and delivery mechanism as recited in claim 1, wherein said container in said upright orientation is suitable for carrying by hand by said user.

6. An apparatus for implementing a versatile liquid storage and delivery mechanism as recited in claim 1, wherein a feeding tube is used when said container is in said inverted orientation, and includes any mechanism that enables liquids to be delivered from said container to a location at which said user can receive liquids.

7. An apparatus for implementing a versatile liquid storage and delivery mechanism as recited in claim 6, wherein said feeding tube includes one or more controlled-delivery devices, wherein said controlled-delivery device includes at least one of a hand-controlled squeezer, a hand-controlled pump, a mechanical controller, or an electronic controller, wherein said controlled-delivery device enables said user or a caregiver of said user to do at least one of control rate of delivery of liquids, monitor delivery of liquids, or meter delivery of liquids.

8. An apparatus for implementing a versatile liquid storage and delivery mechanism as recited in claim 9, wherein a first end of said feeding tube is at or near said opening of said container, wherein a second end of said feeding tube has a mouthpiece, wherein said mouthpiece further includes a flow control provision to prevent escape of liquids when said feeding tube is not in use.

9. An apparatus for implementing a versatile liquid storage and delivery mechanism as recited in claim 1, wherein said container has an additive inlet that enables adding one or more additives to contents of said container, wherein said additive inlet includes at least one of:
a sealable additive opening on the exterior of said container, wherein said sealable additive opening is unsealed to add said additives and said sealable additive opening is sealed after said additives are added to contents of said container; or
an injectable area on the exterior of said container, wherein a needle is pierced through said injectable area to enable said additives to be injected into said container.

10. An apparatus for implementing a versatile liquid storage and delivery mechanism as recited in claim 9, wherein standardized pellets are used for adding said additives to contents of said container, wherein each of said standardized pellets contains a specific quantity of said additives, wherein said standardized pellet includes a pellet opening to allow flow of liquids out of said standardized pellet, said pellet opening including one of a nozzle, wherein said nozzle is adapted to fit within said sealable additive opening of said container, or a needle arrangement that can be used to pierce through said injectable area of said container.

11. A method for implementing a versatile liquid storage and delivery mechanism, the method comprising:
using a container to store or deliver liquids, wherein said container has an upper portion and a lower portion;
using said container interchangeably in an upright orientation or in an inverted orientation, wherein:
using said container in said upright orientation enables a person to directly drink liquids from said container; and
using said container in said inverted orientation includes mounting said container from a support source, enabling said container to be used for at least one of:
oral delivery of liquids; or
enteral delivery of liquids; and
using a placement provision, wherein said placement provision enables said container to be placed on a solid surface in said upright orientation, and wherein said placement provision enables said container to be mounted from said support source in said inverted orientation.

12. A method for implementing a versatile liquid storage and delivery mechanism recited in claim 11, wherein said container is in the shape of a tear drop, wherein said upper portion is tapered and narrow, and said lower portion is rounded and broader than said upper portion, wherein said upper portion points upward and said lower portion points downward when said container is used in said upright orientation, and wherein said upper portion points downward and said lower portion points upward when said container is used in said inverted orientation.

13. A method for implementing a versatile liquid storage and delivery mechanism as recited in claim 11, the method further comprising arranging said containers in a saw-tooth arrangement, wherein in said saw-tooth arrangement:
a first row of said containers is placed in said upright orientation; and
a second row of said containers is placed over said first row of said containers in said inverted orientation such that there is minimal void space between said first row and said second row of said containers.

14. A method for implementing a versatile liquid storage and delivery mechanism as recited in claim 13, wherein said arranging in said saw-tooth arrangement is done when a plurality of said containers are packed in a container, wherein said arranging in said saw-tooth arrangement minimizes damage to said containers, wherein said arranging in said saw-tooth arrangement improves efficiency of shipping said plurality of containers.

15. A method for implementing a versatile liquid delivery mechanism, the method comprising:
using a container interchangeably in an upright orientation or an inverted orientation;
using said container in said upright orientation for delivering liquids to a person, wherein said using said container in said upright orientation enables a person to directly drink liquids from said container;
using said container in said inverted orientation for delivering liquids to a person, wherein using said container in said inverted orientation includes mounting said container on a support source such that said container is suitable for at least one of:
oral delivery of liquids; or
enteral delivery of fluids;
using a feeding provision to enable liquids to be delivered to said person;
using a flow measuring method to measure and quantify delivering of liquids; and
using a flow controlling method to allow said person or a third person to control delivering of liquids.

16. A method for implementing a versatile liquid delivery mechanism as recited in claim 15, wherein said flow measuring method includes using said containers of a specific capacity, wherein said flow measuring method includes using measurement indicators on or within said container.

17. A method for implementing a versatile liquid delivery mechanism as recited in claim 15, wherein said enteral delivery includes one or more of gastric delivery of liquids, or nasogastric delivery of liquids.

18. A method for implementing a versatile liquid delivery mechanism as recited in claim 15, wherein using said container in said inverted position comprises mounting said container on said support source at a height above said person, wherein using said container in said inverted position further comprises delivering liquids by means of gravity through said feeding provision, wherein said mounting said container at a height includes:
mounting said container from an IV stand;
mounting said container from a stand attached to a bed,
mounting said container from a stand attached to a gurney; or
mounting said container from a stand attached to a wheel chair.

19. A method for implementing a versatile liquid delivery mechanism as recited in claim 15, wherein using said container in said upright position includes hand-carrying said container.

20. A method for implementing a versatile liquid delivery mechanism as recited in claim 15, wherein said feeding provision has a flow control provision to prevent escape of liquids when said feeding provision is not used by said person.

21. A method for implementing a versatile liquid delivery mechanism as recited in claim 15, further comprising using an additive inlet to add one or more additives to contents of said container, wherein said adding one or more additives includes at least one of:
adding a base;
adding an acid;
adding a prescriptive or over-the-counter drug;
adding electrolytes;
adding vitamins; or
adding sweeteners.
